(19) United States
(12) Patent Application Publication
(10) Pub. No.: US 2013/0040328 A1
(43) Pub. Date: Feb. 14, 2013

(54) ASSAY FOR PHYTOL-FREE CHLOROPHYLL DERIVATIVES

(71) Applicants: Rene Mikkelsen, Skanderborg (DK); Tina Jorgensen, Silkeborg (DK); Jorn Borch Soe, Tilst (DK)

(72) Inventors: Rene Mikkelsen, Skanderborg (DK); Tina Jorgensen, Silkeborg (DK); Jorn Borch Soe, Tilst (DK)

(73) Assignee: DUPONT NUTRITION BIOSCIENCES APS, 1001 COPENHAGEN K (DK)

(21) Appl. No.: 13/639,915

(22) PCT Filed: Apr. 6, 2011
(86) PCT No.: PCT/IB2011/051474

§ 371 Date: Oct. 8, 2012

(30) Foreign Application Priority Data
Apr. 8, 2010 (EP) 10159327.5

(51) Int. Cl. C12Q 1/44; G01N 21/64

(52) U.S. Cl. 435/19

(57) ABSTRACT

The present invention provides a method for detecting a phytol-free chlorophyll derivative in a sample, comprising a step of detecting a fluorescent signal associated with the phytol-free chlorophyll derivative, wherein a fluorescent signal associated with chlorophyll or a phytol-containing chlorophyll derivative in the sample is quenched. The method may be used for quantifying activity of chlorophyllases and related enzymes in a sample without solvent fractionation of substrate and product.

### FIELD

The present invention relates to a method for detecting chlorophyll derivatives in a sample. The method is useful in an assay for determining the activity of chlorophyllase or related enzymes in a sample.

### BACKGROUND

Chlorophyll is a green-coloured pigment widely found throughout the plant kingdom. Chlorophyll is essential for photosynthesis and is one of the most abundant organic metal compounds found on earth. Thus many products derived from plants, including foods and feeds, contain significant amounts of chlorophyll.

In plants, chlorophyllase (chlase) is thought to be involved in chlorophyll degradation and catalyzes the hydrolysis of an ester bond in chlorophyll to yield chlorophyllide and phytol. Chlorophyll can alternatively be degraded by the loss of the magnesium ion from the porphyrin (chlorin) ring to form the derivative known as pheophytin (see FIG. 5). Under certain conditions, some chlorophyllases are also capable of hydrolyzing pheophytin to yield pheophorbide and phytol. Pheophorbide can also be produced by the loss of a magnesium ion from chlorophyllide, i.e. following hydrolysis of chlorophyll (see FIG. 5).

Pheophytin may be further degraded to pyropheophytin. One possible mechanism is the enzymatic hydrolysis of the methyl ester bond of the isocyclic ring of pheophytin followed by the non-enzymatic conversion of the unstable intermediate to pyropheophytin. A 28-29 kDa enzyme from Chenopodium album named pheophorbidase is reportedly capable of catalyzing an analogous reaction on pheophorbide, to produce the phytol-free derivative of pyropheophytin known as pyropheophorbide (see FIG. 5). Pyropheophytin may also be hydrolyzed by certain enzymes to form pyropheophorbide and phytol.

Various assays have been developed in order to determine chlorophyllase activity in a sample (see e.g. Khamessan et al. (1994), Journal of Chemical Technology & Biotechnology, Vol. 60 (1), pages 73-81). The most widely used method for the determination of chlorophyllase activity is described by Klein and Vishniac in J. Biol. Chem. 1961 236: 2544-2547. This method involves determining enzyme activity in an aqueous buffer system containing acetone. Acetone is added to ensure the solubility of the substrate (chlorophyll). After enzyme reaction has taken place the residual substrate is extracted into hexane. The reaction product chlorophyllide is more hydrophilic than chlorophyll due to loss of the phytol chain. Thus chlorophyllide remains in the water/acetone phase and can be quantified by spectrophotometer measurement. Although various modifications of this method have been described, all published methods involve a step of hexane fractionation of the reaction products. This step is necessary because it is difficult to distinguish between chlorophyll and chlorophyllide using spectroscopic techniques. After hexane extraction, the substrate and reaction product are in different phases and thus measurement of either can be used to determine enzyme activity.

However hexane fractionation is inconvenient, laborious and not well suited for use in a high throughput screening (HTS) method for chlorophyllase activity. An attempt to overcome the problems with hexane extraction in HTS of chlorophyllase was reported by Arkus et al. in Analytical Biochemistry 353 (2006) 93-98. Instead of using chlorophyll as substrate, this assay employs p-nitrophenyl ester as an artificial substrate in HTS of chlorophyllase. Unfortunately this method is not very reliable because some chlorophyllases have rather low activity on p-nitrophenyl ester, despite having high activity on chlorophyll, which may give false negative results. Moreover microbial chlorophyllases are often expressed together with other esterases, which may act on p-nitrophenyl ester but not chlorophyll thus giving false positive results.

Thus there is still a need for an improved assay for chlorophyllase and related enzymes. In particular, there is a need for a method which avoids the disadvantages of solvent extraction and which is reliable, accurate and suitable for high throughput screening.

### SUMMARY

Accordingly, the present invention provides a method for detecting a phytol-free chlorophyll derivative in a sample, comprising a step of detecting a fluorescent signal associated with the phytol-free chlorophyll derivative, wherein a fluorescent signal associated with chlorophyll or a phytol-containing chlorophyll derivative in the sample is quenched.

In one embodiment the fluorescent signal associated with chlorophyll or the phytol-containing chlorophyll derivative is quenched by dimerization of chlorophyll or the phytol-containing chlorophyll derivative. Preferably under the conditions used in the detection step, chlorophyll or the phytol-containing chlorophyll derivative dimerizes preferentially compared to the phytol-free chlorophyll derivative.

In one embodiment the phytol-free chlorophyll derivative comprises chlorophyllide, pheophorbide or pyropheophorbide. In one embodiment chlorophyll or the phytol-containing chlorophyll derivative comprises chlorophyll, pheophytin or pyropheophytin.

The detecting step may be performed in a detection solution comprising a surfactant, an alcohol and an alkali. Preferably the alcohol comprises isopropanol, more preferably 12 to 20% by weight alcohol.

In one embodiment the surfactant is present at 0.01 to 0.03% by weight, based on the total weight of the detection solution, and preferably comprises 4-(1,1,3,3-tetramethylbutyl)phenyl-polyethylene glycol.

In one embodiment the detecting step is performed at 20 to 25° C., and preferably at a pH of greater than 10.0. The fluorescent signal may be detected at, for example, about 670 nm.

In one embodiment chlorophyll or the phytol-containing chlorophyll derivative is present in the form of an aqueous solution of dimers and/or other non-colloidal multimers.

In one embodiment the detection step is performed in the absence of liposomes.

In one embodiment, the surfactant comprises 4-(1,1,3,3-tetramethylbutyl)phenyl-polyethylene glycol, the alcohol comprises isopropanol and the alkali comprises sodium hydroxide.

In another aspect the present invention provides an assay method for quantifying enzyme activity in a sample, wherein the enzyme is capable of hydrolyzing chlorophyll or a phytol-containing chlorophyll derivative, the assay method comprising: a) contacting the sample with chlorophyll or a phytol-containing chlorophyll derivative; and b) detecting production of a phytol-free chlorophyll derivative by a method as described above.

In one embodiment, step (a) comprises incubating the sample with chlorophyll or a phytol-containing chlorophyll derivative in a reaction solution comprising a surfactant, acetone and/or a buffer. Preferably after step (a) and before step (b), enzyme activity is terminated by adding the reaction solution to a detection solution as defined above.

In one embodiment, the enzyme is active during step (a) and the enzyme is inactive during step (b).

In a further aspect, the present invention provides a kit for quantifying enzyme activity in a sample, wherein the enzyme is capable of hydrolyzing chlorophyll or a phytol-containing chlorophyll derivative, the kit comprising: a) a reaction solution in which the enzyme is active; b) a substrate comprising chlorophyll or a phytol-containing chlorophyll derivative; and c) a detection solution in which the substrate dimerizes preferentially compared to a phytol-free chlorophyll derivative produced by the enzyme.

In one embodiment, the reaction solution comprises a surfactant, acetone and a buffer. Preferably the substrate comprises chlorophyll, pheophytin or pyropheophytin. The detection solution may be a detection solution as described above. Preferably the enzyme is inactive in the detection solution.

In one embodiment the kit further comprises one or more standard solutions, each comprising a known concentration of the phytol-free chlorophyll derivative.

### BRIEF DESCRIPTION OF DRAWINGS

FIG. 1 shows formation of a dimer from two molecules of pheophytin. Dimerization occurs via the porphyrin (chlorin) ring and leads to quenching of fluorescence at 670 nm.

FIG. 2 shows a standard (calibration) curve of relative fluorescence (RFU) against pyropheophorbide concentration (μM).

FIG. 3 shows a standard (calibration) curve of relative fluorescence (RFU) against pheophorbide concentration (μM).

FIG. 4 shows the hydrolysis of pheophytin resulting in production of pheophorbide and phytol, catalysed by pheophytinase.

FIG. 5 shows reactions involving chlorophyll and derivatives and enzyme activities which may be detected using the present invention.

### DETAILED DESCRIPTION

In one aspect the present invention relates to a method for detecting a phytol-free chlorophyll derivative in a sample. By quenching a fluorescent signal derived from chlorophyll or phytol-containing chlorophyll derivatives in the sample, the phytol-free chlorophyll derivative can be directly detected. The method can be quantitative, i.e. the method can be used to determine a level or concentration of the phytol-free chlorophyll derivative in the sample. The method is therefore particularly useful for determining chlorophyllase or related enzyme activity in a sample, since it enables product to be distinguished from substrate without physical separation (i.e. without requiring hexane extraction or other solvent fractionation of substrate and product).

### Chlorophyll and Chlorophyll Derivatives

By “phytol-containing chlorophyll derivative” it is typically meant compounds which comprise both a porphyrin (chlorin) ring and a phytol group (tail), including magnesium-free phytol-containing derivatives such as pheophytin and pyropheophytin. Chlorophyll and phytol-containing chlorophyll derivatives are typically greenish is colour, as a result of the porphyrin (chlorin) ring present in the molecule. Preferably the chlorophyll or phytol-containing chlorophyll derivative is chlorophyll, pheophytin or pyropheophytin, more preferably pheophytin or pyropheophytin. Chlorophyll or a phytol-containing chlorophyll derivative is typically used as the substrate in an assay method as described herein.

By “phytol-free chlorophyll derivative” it is typically meant the product of enzymatic hydrolysis of a phytol-containing chlorophyll derivative. Chlorophyllase or related enzymes may hydrolyse chlorophyll and phytol-containing chlorophyll derivatives to cleave the phytol tail from the chlorin ring. These compounds still contain the colour-bearing porphyrin ring, with chlorophyllide being green and pheophorbide and pyropheophorbide a reddish brown colour. Preferably the phytol-free chlorophyll derivative is chlorophyllide, pheophorbide or pyropheophorbide, more preferably pheophorbide or pyropheophorbide. A phytol-free chlorophyll derivative is typically the reaction product in an assay method as described herein.

The chlorophyll or chlorophyll derivative may be, for example, a, b or d forms. Thus as used herein, the term “chlorophyll” includes chlorophyll a, chlorophyll b and chlorophyll d. In a similar way a, b and d forms are covered when referring to pheophytin, pyropheophytin, chlorophyllide, pheophorbide and pyropheophorbide.

The detection method as described herein typically permits the discrimination of chlorophyll or a phytol-containing chlorophyll derivative from its corresponding phytol-free chlorophyll derivative. For example, the method may enable a substrate and a reaction product of a chlorophyllase or related enzyme to be distinguished from one another. In particular embodiments, the substrate/product (phytol-containing/phytol-free) pairs may be (a) chlorophyll and chlorophyllide; (b) pheophytin and pheophorbide; or (c) pyropheophytin and pyropheophorbide.

### Sample

The present method may be used to detect a phytol-free chlorophyll derivative present in a sample. The sample may, for example, comprise a plant-derived preparation, an algal preparation or a bacterial-derived preparation, derived from any type of plant, algae or bacteria (e.g. cyanobacteria). In one embodiment the sample comprises a plant material, plant oil or plant extract. For instance, the sample may comprise a plant oil such as a vegetable oil, including oils processed from oil seeds or oil fruits (e.g. seed oils such as canola (rapeseed) oil and fruit oils such as palm).

As described herein, in some embodiments the method is used to assay for enzyme (e.g. chlorophyllase, pheophytinase and/or pyropheophytinase) activity in a sample. In such embodiments, the sample may comprise any preparation suspected to contain the relevant enzyme activity. The sample may, for example, comprise a plant, algal or bacterial preparation or extract or may comprise a purified or recombinant protein to be tested for chlorophyllase or a related enzyme activity. In these embodiments, phytol-free chlorophyll derivatives may be absent from the sample prior to the contacting step of the assay method, i.e. phytol-free derivatives may be produced following addition of a suitable substrate for the enzyme.

### Detecting a Fluorescent Signal

In embodiments of the present invention, a fluorescent signal associated with a phytol-free chlorophyll derivative is detected. For example, a fluorescent signal derived from, or emitted by, the phytol-free derivative may be detected, measured or quantified, preferably in the absence of a fluorescent signal derived from chlorophyll or a phytol-containing chlorophyll derivative.

Methods for detecting fluorescent signals are well known in the art. For example, the fluorescent signal derived from the phytol-free chlorophyll derivative may be detected by any suitable detector, e.g. a fluorescent spectrophotometer, fluorescent plate reader or spectrofluorometer. The detector typically includes a light source that produces light at an appropriate wavelength for activating the fluorescent material, as well as optics for directing the light source through a detection window to the material contained in the channel or chamber. Various light sources may be used as excitation sources, including lasers, photodiodes, and xenon or mercury-vapor lamps. The light may be passed through a filter (e.g. a diffraction grating) or monochromator in order to select a fixed wavelength before passing through the sample. Emitted light may be passed through a filter or a monochromator and a specific wavelength detected by a photodetector typically at 90 degrees to the excitation light. The photodetector may comprise, for example, a photomultiplier tube, photodiode, or charge-coupled device (CCD) detector. The photodetector typically provides a value for the intensity of the fluorescent signal. The signal can either be processed as a digital or analog output.

In embodiments of the present invention, excitation and emission wavelengths suitable for fluorescent detection of chlorophyll derivatives are selected, e.g. excitation at about 410 nm and emission at about 670 nm. Typically the intensity of the fluorescent signal detected is proportional to the concentration of the phytol-free chlorophyll derivative in the sample.

Quenching a Fluorescent Signal from Chlorophyll or Phytol-Containing Chlorophyll Derivatives

In embodiments of the present invention, a fluorescent signal associated with chlorophyll or a phytol-containing chlorophyll derivative in the sample is quenched. By “quenched” it is meant that the intensity of a fluorescent signal derived from chlorophyll or a phytol-containing chlorophyll derivative in the sample is decreased, inhibited or suppressed. In a preferred embodiment, the fluorescent signal associated with chlorophyll or the phytol-containing chlorophyll derivative is quenched by dimerization. By “dimerization” it is meant that at least two molecules of the phytol-containing chlorophyll derivative associate to form at least a dimer. Thus “dimerization” as used herein includes the formation of higher order structures comprising three or more molecules of the phytol-containing chlorophyll derivative, such as trimers, tetramers or other multimers, provided that the fluorescent signal from such structures is quenched, and provided that the dimers or other multimers remain in solution (e.g. the dimers or multimers are solutes in an aqueous solution). By this it is meant that typically the chlorophyll or phytol-containing chlorophyll derivative does not form solid particles in the detection solution, e.g. the chlorophyll or phytol-containing chlorophyll derivative does not precipitate, or does not form solid colloidal aggregates in the detection solution. “Dimerization” and “multimerization” may thus be used interchangeably and “dimerizes”, “dimerized”, “dimerizing” and “dimer formation” should also be construed accordingly.

It is known from HPLC analysis (Food Research International 38 (8-9): 1067-1072 (2005)) that phytol-containing compounds such as chlorophyll, pheophytin and pyropheophytin give a very strong fluorescence signal by excitation at 410 nm and emission at 667 nm. Under certain conditions the porphyrin ring of chlorophyll, pheophytin and pyropheophytin is capable of forming a dimer, which strongly quenches the fluorescence signal (see J. Photochem. Photobiol. B: Biol. 54 (2000) 194-200). Phytol-free chlorophyll derivatives such as chlorophyllide, pheophorbide and pyropheophorbide are also capable of dimerizing via the porphyrin ring, resulting in a similar decrease in fluorescence intensity. However, the conditions under which phytol-containing and phytol-free chlorophyll derivatives dimerize may differ. In embodiments of the present invention, this difference may be exploited in order to select conditions under which fluorescence is detected exclusively from the phytol-free derivatives.

Thus in one embodiment, the detecting step is performed under conditions such that chlorophyll or the phytol-containing chlorophyll derivative dimerizes preferentially compared to the phytol-free chlorophyll derivative. By “dimerizes preferentially” it is meant that the proportion of chlorophyll or the phytol-containing chlorophyll derivative which is dimerized is greater than the proportion of the phytol-free chlorophyll derivative which is dimerized under those conditions.

In some embodiments, at least 50%, at least 70%, at least 90% or at least 95% of the fluorescent signal derived from chlorophyll or the phytol-containing chlorophyll derivative is quenched, preferably by dimerization. By this, it is meant that under the detection conditions used, the intensity of the fluorescent signal derived from the chlorophyll or phytol-containing chlorophyll derivative is reduced by the specified proportion compared to a fluorescent signal derived from chlorophyll or the phytol-containing chlorophyll derivative under control conditions in which no quenching occurs, e.g. under conditions where there is no significant dimerization of chlorophyll or the phytol-containing chlorophyll derivative. For example, non-quenching conditions may comprise a high (e.g. >0.1%) concentration of surfactant.

Preferably the fluorescent signal derived from the phytol-free chlorophyll derivative is not significantly quenched, or is quenched to a lesser degree than the fluorescent signal derived from chlorophyll or the phytol-containing chlorophyll derivative. For example, in some embodiments less than 50%, less than 25% or less than 10% of the fluorescent signal derived from the phytol-free chlorophyll derivative is quenched compared to a fluorescent signal derived from the phytol-free chlorophyll derivative under control conditions in which no quenching occurs, e.g. under conditions where there is no significant dimerization of the phytol-free chlorophyll derivative.

### Detection Conditions

The detecting step of the present invention may be performed under conditions such that a fluorescent signal associated with chlorophyll or a phytol-containing chlorophyll derivative in the sample is quenched, preferably by dimerization. By varying the composition of a solution in which the signal is detected, a skilled person can select appropriate conditions in order to quench the fluorescent signal, e.g. conditions under which the chlorophyll or a phytol-containing chlorophyll derivative is predominantly dimerized but the phytol-free chlorophyll derivative is not dimerized.

Typically the detecting step is performed in an aqueous solution. The solution in which the detecting step is performed is referred to herein as a “detection solution”. It has been found that the composition of the detection solution can be varied in order to influence dimerization of chlorophyll derivatives and quenching of the fluorescent signal. In particular, the concentration of surfactant, temperature, pH, and type and concentration of solvent in the detection solution may influence dimerization and can be varied in order to select suitable conditions for the detection step. Preferably the detection solution comprises a surfactant, a solvent and/or an alkali.

The solvent is typically a polar protic solvent such as an alcohol, preferably a lower alcohol (e.g. C₁-C₅ or C₁-C₃), more preferably an aliphatic monohydric alcohol. In particular embodiments, the alcohol comprises methanol, ethanol, propanol, isopropanol or butanol, more preferably ethanol or isopropanol, most preferably isopropanol. The detection solution may comprise 5 to 25% by weight alcohol, e.g. 5 to 20%, 5 to 15%, 10 to 20%, 12 to 20%, 13 to 17% or about 15% by weight alcohol, e.g. ethanol or isopropanol.

An appropriate concentration of the solvent (e.g. alcohol) may be selected based on the specific alcohol and the other conditions, i.e. surfactant concentration, pH, temperature etc. If other reaction conditions are kept constant, increasing the solvent concentration typically reduces dimerization and therefore reduces quenching of the fluorescent signal. In some embodiments, the solvent concentration may be titrated (e.g. progressively increased whilst maintaining other conditions constant) until a concentration is reached at which the phytol-free chlorophyll derivative no longer dimerizes, but at which the chlorophyll or phytol-containing derivative remains in a dimerized state. At this point, the fluorescent signal from the chlorophyll or phytol-containing derivative remains quenched but the signal from the phytol-free derivative is not quenched. This concentration of the solvent may then be used in the detecting step.

The nature of the surfactant is not particularly limited. In particular embodiments, the surfactant may be, for example, an anionic surfactant, a cationic surfactant, a Zwitterionic surfactant, or a nonionic surfactant.

Suitable anionic surfactants include, for example, carboxylates, such as soaps and polyalkoxycarboxylates; sulfonates, such as alkylbenzenesulfonates, alkylarenesulfonates, napthalenesulfonates, α-olefinsulfonates, sulfonates with ester, amide, or ether linkages including amidosulfonates, 2-sulfoethyl esters of fatty acids, and fatty acid ester sulfonates; sulfates, such as alcohol sulfates, ethoxylated and sulfated alcohols, ethoxylated and sulfated alkylphenols, sulfated acids, sulfated amides, sulfated esters, and sulfated natural oils and fats; phosphate esters, such as butyl phosphate, hexyl phosphate, 2-ethylhexyl phosphate, octyl phosphate, decyl phosphate, oxtyldecyl phosphate, mixed alkyl phosphate, hexyl polyphosphate, octyl polyphosphate, glycerol monoester of mixed fatty acids (phosphated), 2-ethylhexanol (ethoxylated and phosphated), deodecyl alcohol (ethoxylated and phosphated), tridecyl alcohol (branched), 9-octadecenyl alcohol (ethoxylated and phosphated), polyhydric alcohols (ethoxylated and phosphated), phenol (ethoxylated and phosphated), octylphenol (ethoxylated and phosphated), nonylphenol (ethoxylated and phosphated), dodecylphenol (ethoxylated and phosphated), and dinonylphenol (ethoxylated and phosphated); and phosphonate esters.

Suitable cationic surfactants include, for example, amines, such as oxygen-free amines including mono-, di-, and polyamines, oxygen-containing amines including amine oxides, ethoxylated alkylamines, 1-(2-hydroxyethyl)-2-imidazolines, and alkoxylates of ethylenediamine, ethylenediamine alkoxylates, and amines with amide linkages; and quaternary ammonium salts, such as dialkyldimethylammonium salts, alkylbenzyldimethylammonium chlorides, alkyltrimethyl ammonium salts, alkylpyridinium halides, and quaternary ammonium esters.

Examples of Zwitterionic surfactants include alkylbetaines, amidopropylbetaines, alkyldimethylamines, imidazolinium derivatives, and amino acids and their derivatives.

Nonionic surfactants which may be used include carboxylic acid esters, such as glycerol esters and polyoxyethylene esters; anhydrosorbitol esters, such as ethoxylated anhydrosorbitol esters; polyoxyethylene surfactants, such as alcohol ethoxylates and alkylphenol ethoxylates; natural ethoxylated fats, oils and waxes; glycol esters of fatty acids; alkyl polyglycosides; carboxylic amides, such as diethanolamine condensates, monoalkanolamine condensates including coco, lauric, oleic, and stearic monoethanolaimdes and monoisopropanolamides, polyoxyethylene fatty acid amides; fatty acid glucamides; and polyoxyalkylene block copolymers.

Many suitable surfactants are available commercially, including: polyoxyethylene-polyoxypropylene block co-polymers of the Pluronic® family; polyethyleneglycol-hydrogenated castor oils available under the trade name Cremophor®, e.g. Cremophor® RH 40; products available under the trade name Nikkol® (e.g. Nikkol® HCO-40 and HCO-60); polyoxyethylene-glycerol-fatty acid esters available under the name Tagat® (e.g. Tagat® RH 40; and Tagat® TO); polyoxyethylene-sorbitan-fatty acid esters, for example mono- and tri-lauryl, palmityl, stearyl and oleyl esters of the type commercially available under the trade name Tween® (e.g. Tween® 20, Tween® 40 or Tween® 80); phospholipids, in particular lecithins such as soya bean lecithins; sorbitan fatty acid esters commercially available under the trade mark Span® e.g. Span® 20 (sorbitan monolaurate) or Span® 80 (sorbitan monooleate).

In one embodiment, the surfactant is a polyoxyethylene surfactant, preferably having a polymerization number of the polyoxyethylene moiety from about 5 to about 50, e.g 8 to 12. More preferably the surfactant comprises an alkaryl ethoxylate, such as an alkylphenol ethoxylate, e.g. octylphenol polymerized with ethylene oxide. In one embodiment the surfactant comprises 4-octylphenol polyethoxylate (4-(1,1,3,3-tetramethylbutyl)phenyl-polyethylene glycol), e.g. available under the trade name Triton X-100 from Sigma-Aldrich, Saint Louis, Mo.

Preferably the detection solution comprises 0.01 to 0.1% by weight, e.g. 0.01 to 0.05%, 0.01 to 0.04%, 0.01 to 0.03% or 0.01 to 0.02% by weight surfactant. An appropriate concentration of the surfactant may be selected based on the specific surfactant and the other conditions, i.e. solvent concentration, pH, temperature etc. If other reaction conditions are kept constant, increasing the surfactant concentration typically reduces dimerization and therefore leads to an increase in the fluorescent signal. In some embodiments, the surfactant concentration may be titrated (e.g. progressively increased whilst maintaining other conditions constant) until a concentration is reached at which the phytol-free chlorophyll derivative no longer dimerizes, but at which the chlorophyll or phytol-containing derivative remains in a dimerized state. This concentration of the surfactant may then be used in the detecting step.

The detecting step may be performed at any suitable temperature. However, if other reaction conditions are kept constant, increasing temperature typically reduces dimerization and therefore reduces quenching of the fluorescent signal. Thus the reaction temperature may be selected such that under the other conditions used (e.g. solvent and surfactant concentration, pH etc.), the chlorophyll or phytol-containing derivative dimerizes preferentially compared to the phytol-free chlorophyll derivative. In particular embodiments, the detecting step may be performed at 10 to 50° C., 15 to 40° C., 15 to 30° C. or preferably at room temperature, e.g. 20 to 25° C.

The detecting step may be performed at any pH. Preferably the detecting step is performed at an alkaline pH, i.e. at a pH of greater than 7, preferably 8.0 or above, 9.0 or above, 10.0 or above or 11.0 or above. A desired pH may be achieved by adding a suitable amount of an alkali, e.g. sodium hydroxide, to the detection solution.

In one embodiment, the detection solution does not comprise liposomes. Advantageously, the detection method of the present invention may be performed without the need for liposomes to enhance fluorescence from the phytol-free chlorophyll derivative.

### Quantifying Phytol-Free Chlorophyll Derivative Levels in the Sample

Because fluorescence from chlorophyll or phytol-containing chlorophyll derivatives is quenched in the detection step, fluorescence intensity values can be equated to a particular level or concentration of phytol-free chlorophyll derivatives in the sample. Typically a standard (calibration) curve is produced adding a known concentration of a phytol-free chlorophyll derivative (e.g. chlorophyllide, pheophorbide or pyropheophorbide) and obtaining fluorescence values. Fluorescence values from samples containing unknown amounts of the phytol-free chlorophyll derivative can then be compared to the standard curve to provide a value for the concentration of the phytol-free chlorophyll derivative in the sample.

### Assay Method

In one aspect of the present invention, the detection method described above may be used in an assay method for quantifying enzyme activity in a sample. In particular, the method may be used to measure enzymatic hydrolysis of chlorophyll or a phytol-containing chlorophyll derivative, by monitoring the production of phytol-free chlorophyll derivatives.

Any enzyme which is capable of hydrolysing chlorophyll or a phytol-containing chlorophyll derivative may be assayed using this method. Typically “hydrolyzing chlorophyll or a phytol-containing chlorophyll derivative” means hydrolysing an ester bond in chlorophyll or a phytol-containing chlorophyll derivative, e.g. to cleave a phytol group from the chlorin ring in the chlorophyll or chlorophyll derivative. Thus the enzyme typically has an esterase or hydrolase activity. The enzyme may, for example, be a chlorophyllase, pheophytinase or pyropheophytinase.

Typically the assay method comprises a step of contacting the sample with chlorophyll or a phytol-containing chlorophyll derivative. Thus the sample may be contacted with a substrate for the enzyme activity which it is desired to detect. For instance, in specific embodiments the sample may be contacted with the following substrates: chlorophyll (in order to detect chlorophyllase activity), pheophytin (in order to detect pheophytinase activity) or pyropheophytin (in order to detect pyropheophytinase activity). The method may also be used to detect any combination of the above activities by adding more than one substrate.

The contacting step may be performed by incubating the sample with chlorophyll or a phytol-containing chlorophyll derivative in a reaction solution. By “reaction solution” it is meant any solution in which the enzyme in the sample is allowed to act on the substrate, i.e. prior to the detecting step. The sample may be added directly to the reaction solution and substrate, followed by incubation for a predetermined period of time in which enzymatic activity takes place.

The contacting step may be performed under any conditions in which the enzyme has hydrolytic activity. Conditions under which chlorophyllases and related enzymes are active are described with reference to known assay methods in, for example, Khamessan et al. (1994), Journal of Chemical Technology & Biotechnology, 60 (1), pages 73-81; Khamessan et al. (1996), Journal of Biotechnology, 45 (3) pages 253-264; Klein and Vishniac (1961), J. Biol. Chem. 236: 2544-2547; McFeeters et al., Plant Physiology 47:609-618 (1971); and McFeeters et al., Plant Physiology 55:377-381 (1975).

Preferably the reaction solution comprises a surfactant, a solvent and/or a buffer. The surfactant and solvent contribute to solubilizing the substrate (e.g. chlorophyll, pheophytin or pyropheophytin) in the solution. The surfactant may be any surfactant as described above in relation to the detection solution, e.g. a polyoxyethylene surfactant such as Triton X-100. However, the reaction solution typically comprises a higher concentration of surfactant than is present in the detection solution. For example, the reaction solution may comprise 0.05 to 0.5%, 0.1 to 0.5% or 0.1 to 0.3% by weight of surfactant.

The reaction solution may comprise an organic solvent such as acetone, ethanol, propanol, butanol or the like. The activity of chlorophyllases in various organic solvents is described, for example, in Khamessan et al. (1995) Process Biochemistry 30 (2), pages 159-168. Preferably the reaction solution comprises acetone as solvent. In some embodiments the reaction solution comprises 1 to 10%, e.g. 3 to 7% or about 5% by weight acetone.

The reaction solution may comprise any suitable buffer. Buffers which may be used include, for example, phosphate or HEPES (4-2-hydroxyethyl-1-piperazineethanesulfonic acid) buffers. Preferably the reaction solution has a pH in the range 6.0 to 8.0, e.g. 6.5 to 7.5 or about 7, and a skilled person can easily select a buffer appropriate for the desired pH. The reaction solution may comprise one or more further salts such as potassium chloride.

The sample may be incubated with the substrate and reaction solution for a suitable period of time in order to allow hydrolysis of a detectable amount of product. Typical incubation times include, for example, 10 seconds to 120 minutes, e.g. 1 to 60 minutes, 1 to 30 minutes, or 5 to 20 minutes. The incubation step may be performed at any temperature at which the enzyme is active, e.g. at 10 to 50° C., 15 to 45° C., 20 to 25° C., or 35 to 45° C., preferably about 40° C.

After the contacting step, enzymatic activity is typically terminated before the fluorescent signal is detected. Enzymatic activity may be terminated by, for example, increasing the pH of the solution, preferably to at least pH 10 or at least pH 11. In one embodiment, an alkali (e.g. sodium hydroxide) is added after the incubation step in order to stop the enzyme reaction. Conveniently, enzyme activity may be terminated by adding the reaction solution to a detection solution as described above, e.g. wherein the detection solution comprises an alkali such as sodium hydroxide. Typically the reaction solution is diluted at least 5 fold, 10 fold, 50 fold, or 100 fold into the detection solution.

Following the enzymatic reaction, the assay method comprises a step of detecting production of a phytol-free chlorophyll derivative by a method as described above. Typically the concentration of the phytol-free chlorophyll derivative in the detection solution is determined. In this manner, a rate of hydrolysis of the substrate per unit time (in the contacting step) can be determined, which provides a measure of the enzyme activity in the sample. One unit of enzyme activity may be defined as the amount of enzyme which hydrolyzes one micromole of substrate (e.g. chlorophyll, pheophytin or pyropheophytin) per minute at 40° C., e.g. in an assay method as described herein. Since substrate and product are equimolar, one unit may alternatively be defined as the amount of enzyme which produces one micromole of product (e.g. chlorophyllide, pheophorbide or pyropheophorbide) per minute at 40° C.

Advantageously, as described above the assay method of the present invention comprises two discrete steps, a reaction step in which the enzyme is active, and a detection step in which the enzyme is inactive. Because the reaction and detection steps take place separately, they can be performed in different solutions and under different conditions. This allows the conditions for each step to be optimized. Thus in one embodiment the enzyme is active in the reaction solution, but is inactive in the detection solution. For instance, the reaction step may be performed using any conditions at which the enzyme is active, e.g. at pH 4 to 8. In contrast, the detection step may be performed using conditions which are optimized to favour selective dimerization of chlorophyll or the phytol-containing chlorophyll derivative, but at which the enzyme is inactive, e.g. at pH 10 or greater.

### Kits

In a further aspect, the present invention provides kits suitable for performing a method or assay method as described herein. Such kits may comprise reagents as described herein for use in the methods, in particular reaction solutions, detection solutions and substrates as described herein. Each component may be contained in suitable vials or other containers together with suitable packaging and/or instructions for performing the method.

The reaction solution is typically any solution in which the enzyme is active. The reaction solution may comprise a surfactant, solvent and a buffer, or may be any other reaction solution described above in relation to the assay method.

Preferably the substrate comprises chlorophyll or a phytol-containing chlorophyll derivative such as chlorophyll, pheophytin or pyropheophytin. One, two or three or more substrates may be provided in the kit, in separate containers or in combination. For instance, in some embodiments two or more substrates may be present in the same container, e.g. where it is desired to assay for two or more enzyme activities. Typically the substrate is provided in the form of an aqueous solution. In some embodiments, the reaction solution and substrate may be provided in a single solution.

The kit may comprise any detection solution as described herein. Preferably fluorescence from chlorophyll or a phytol-containing chlorophyll derivative is quenched in the detection solution, whereas fluorescence from the phytol-free chlorophyll derivative is not quenched, e.g. the detection solution is selected such that the substrate dimerizes preferentially compared to the product of enzymatic hydrolysis. In one embodiment, the detection solution comprises a surfactant, an alcohol (e.g. isopropanol) and an alkali (e.g. sodium hydroxide). Typically the enzyme is inactive in the detection solution.

In a preferred embodiment, the kit further comprises one or more standards, e.g. for constructing a standard (calibration) curve for use in the assay method. Typically such standards are in the form of containers each containing a known concentration of the product, i.e. a phytol-free chlorophyll derivative. One, two or more standards may be provided, containing different products (e.g. chlorophyllide, pheophorbide or pyropheophorbide) and/or differing concentrations of each product.

The invention will now be further illustrated with reference to the following non-limiting examples.

### EXAMPLES

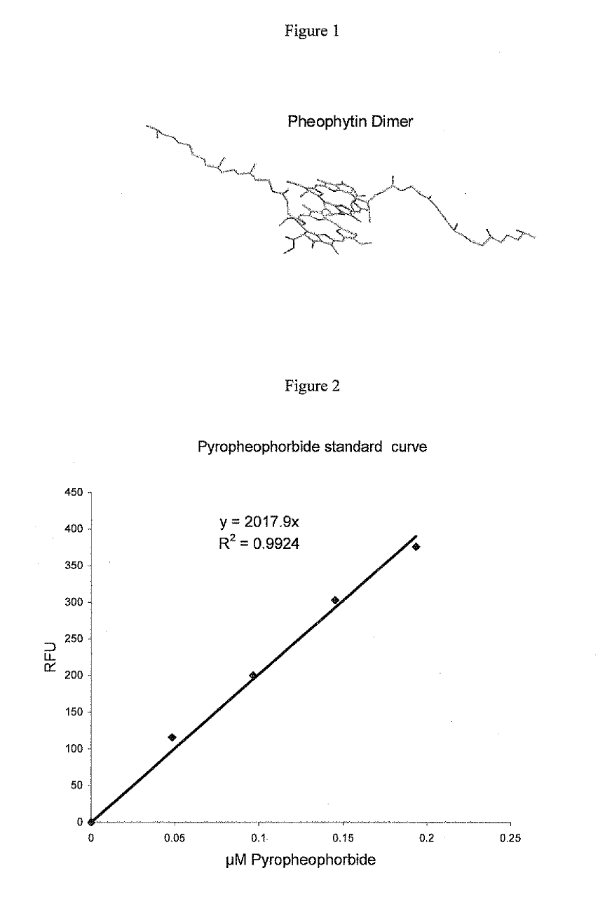

In these examples, an assay method for measurement of chlorophyllase, pheophytinase and pyropheophytinase activity was developed. In the first step of the assay a substrate composed of chlorophyll, pheophytin or pyropheophytin in a buffer was prepared. This substrate was added to a solution of enzyme and reacted for 10 minutes at 40° C. After 10 minutes reaction time part of the sample was transferred to a detection buffer. Measurement of enzyme activity relies on the fact that chlorophyll, pheophytin or pyropheophytin in the detection buffer forms a dimer (see FIG. 1). The dimer formation quenches the fluorescence signal of chlorophyll, pheophytin or pyropheophytin. The detection buffer is formulated such that chlorophyllide, pheophorbide or pyropheophorbide produced does not form a dimer and thus can be detected by fluorescence spectroscopy.

### Example 1

### Development of an Assay for Pyropheophytinase Activity

A 100 mM phosphate buffer, pH 7, containing 50 mM KCl, 0.2% Triton X-100 and 5.17% acetone was prepared for use as the reaction buffer. The following solutions were prepared, comprising pyropheophytin, pyropheophorbide or a mixture thereof, and added to the reaction buffer:

1) Pyropheophytin solution: 500 μl reaction buffer+50 μl water+30 μl pyropheophytin (1 mg/ml in acetone)

2) Pyropheophorbide solution: 500 μl reaction buffer+50 μl water+15 μl pyropheophorbide (2 mg/ml in acetone)+15 μl acetone.

3) Pyropheophytin:Pyropheophorbide 1:1 solution: 500 μl reaction buffer+50 μl water+15 μl Pyropheophytin (1 mg/ml in acetone)+7.5 μl pyropheophorbide (2 mg/ml in acetone)+7.5 μl acetone.

Various detection solutions (A-G) comprising differing concentrations of Triton X-100 (surfactant) and ethanol or isopropanol (solvent) were prepared, as shown in Tables 1 and 2 below. 10 μl of solution 1, 2 or 3 was added to 1000 μl of each detection solution A-G, mixed on a Whirley mixer and 200 μl transferred to a Black microliter plate. 10 minutes after mixing the fluorescence signal (excitation 410 nm, emission 670 nm) was measured at room temperature.

The measurements were analysed in order to determine conditions under which fluorescence from the substrate (chlorophyll, pheophytin or pyropheophytin) was quenched but the fluorescence from the reaction product (chlorophyllide, pheophorbide or pyropheophorbide) was not quenched and could be measured by fluorescence measurement.

Initially combinations of ethanol, Triton X-100 and 50 mM NaOH were tested as the detection solution. The results are shown below in Table 1:

TABLE 1

| Detection | | 1 | 2 | 3 |
|---|---|---|---|---|
| solution | Composition | Pyropheophytin | Pyropheophorbide | Mixture |
| A | 100 mM | 1513 | 2132 | 1606 |
| | Phosphate | | | |
| | buffer | | | |
| | 0.2% Triton | | | |
| | X-100 | | | |
| | 50 mM KCl | | | |
| B | 0.03% Triton | 43 | 214 | 209 |
| | X-100 | | | |
| | 10% EtOH | | | |
| | 0.05M NaOH | | | |
| C | 0.05% Triton | 894 | 1800 | 1288 |
| | X-100 | | | |
| | 10% EtOH | | | |
| | 0.05M NaOH | | | |
| D | 0.04% Triton | 455 | 777 | 718 |
| | X-100 | | | |
| | 10% EtOH | | | |
| | 0.05M NaOH | | | |

The values in Table 1 are relative fluorescence (RFU) values, excitation 410 nm and emission 670 nm, for each detection solution A-D in combination with each of solutions 1, 2 and 3.

The results in Table 1 indicate that the concentration of the surfactant (Triton X-100) is very important for the signal. In detection solution B, the fluorescence signal of pyropheophytin is almost eliminated because pyropheophytin forms a dimer, which leads to the fluorescence quenching. The signal for pyropheophorbide is however also strongly quenched. The signal in detection solutions C and D is less quenched for both pyropheophytin and pyropheophorbide, and therefore also detection solutions C and D are not suitable for discrimination of the signal from pyropheophytin and pyropheophorbide.

Next isopropanol (IPA) was used instead of ethanol with results shown in Table 2:

TABLE 2

| Detection | | 1 | 2 | 3 |
|---|---|---|---|---|
| solution | Composition | Pyropheophytin | Pyropheophorbide | Mixture |
| E | 0.015% Triton | 26 | 1185 | 617 |
| | X-100; | | | |
| | 15% IPA; | | | |
| | 0.05M NaOH | | | |
| F | 0.02% Triton | 33 | 431 | 284 |
| | X-100; | | | |
| | 10% IPA; | | | |
| | 0.05M NaOH; | | | |
| G | 0.02% Triton | 26 | 1293 | 639 |
| | X-100 | | | |
| | 15% IPA | | | |
| | 0.05M NaOH | | | |

Figures are RFU values, 410 nm/670 nm

The results in Table 2 indicate a much stronger quenching of pyropheophytin in the presence of isopropanol, but pyropheophorbide gives a very high fluorescence signal. The pyropheophytin and pyropheophorbide mixture (3) gives almost the half signal of pyropheophorbide, which confirm that it is possible to discriminate these two components.

Based on the results above, it was decided to use detection solution E in an assay for pyropheophytinase activity. In order to analyse the amount of pyropheophorbide produced by an enzyme reaction, a calibration curve was constructed by adding varying amounts of pyropheophorbide to detection solution E and measuring fluorescence as described above. The calibration curve is illustrated in FIG. 2.

### Example 2

### Development of an Assay for Pheophytinase Activity

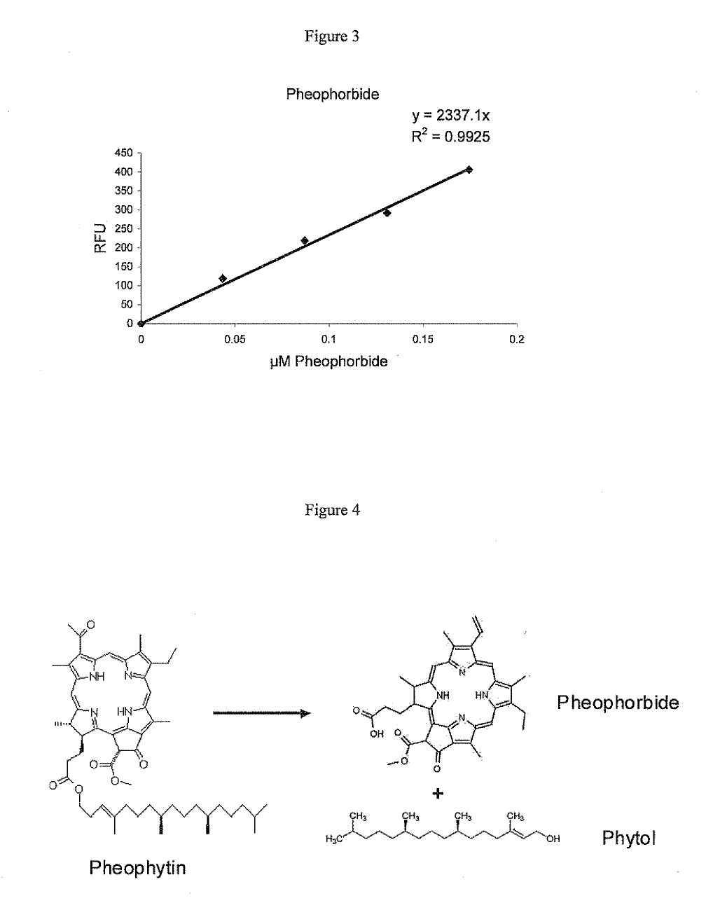

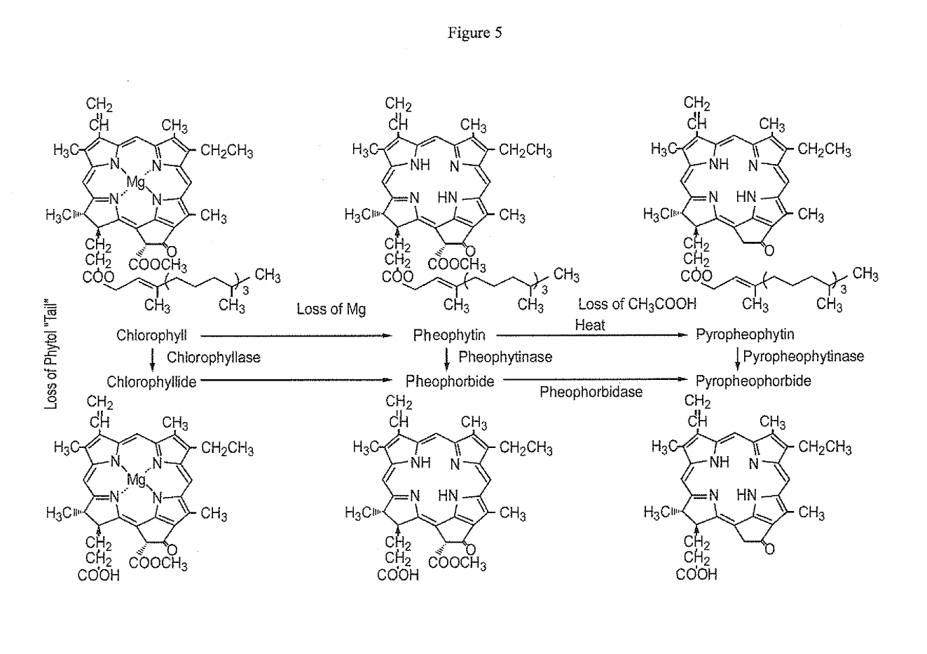

Based on the results of Example 1, an assay for pheophytinase activity was developed. When added to detection solution E (0.015% Triton X-100; 15% IPA; 0.05M NaOH), fluorescence from pheophytin was largely quenched (as for pyropheophytin) but pheophorbide gave a high fluorescence signal at room temperature. Accordingly, a calibration curve was constructed for pheophorbide (see FIG. 3).

### Example 3

### Development of an Assay for Chlorophyllase Activity

Based on the results of Example 1, an assay for chlorophyllase activity was developed.

Using a reaction solution as described in Example 1, the following solution was prepared:

4) Chlorophyll solution: 500 μl reaction buffer+50 μl water+30 μl chlorophyll (1 mg/ml in acetone).

Detection solutions A, B and E as described in Example 1 were prepared. 10 μl of chlorophyll solution 4 was added to 1000 μl of each detection solutions A, B and E, mixed and fluorescence measured at room temperature as described in Example 1. The results are shown in Table 3 below:

TABLE 3

| Detection | | 4 |
|---|---|---|
| solution | Composition | Chlorophyll |
| A | 100 mM Phosphate buffer | 1206 |
| | 0.2% Triton X-100 | |
| | 50 mM KCl | |
| E | 0.02% Triton X-100; | 5 |
| | 15% IPA; | |
| | 0.05M NaOH; | |
| B | 0.03% Triton X-100 | 7 |
| | 10% EtOH | |
| | 0.05M NaOH | |

Figures are RFU values, 410 nm/670 nm

Table 3 shows that chlorophyll gives a high fluorescence signal in the reaction buffer (detection solution A) but the signal is quenched when diluting into detection solutions E and B.

In contrast, chlorophyllide showed a high fluorescence signal in detection solution E.

Based on the above observations, detection solution E (0.015% Triton X-100; 15% isopropanol; 0.05M NaOH) was selected for use in assays for chlorophyllase, pheophytinase and pyropheophytinase activity. In detection solution E, chlorophyllide, pheophorbide and pyropheophorbide each generate high fluorescent signals whereas the signals from chlorophyll, pheophytin and pyropheophytin are quenched.

### Example 4

### Pyropheophytinase Assay

A 100 mM phosphate buffer, pH 7, containing 50 mM KCl, 0.2% Triton X-100 and 5.17% acetone is used as reaction buffer. The substrate (pyropheophytin) is added to the reaction buffer at a concentration of 56 mM. 130 μl of the reaction buffer comprising substrate is transferred to an Eppendorf tube and placed in the in an incubator at 40° C. for 5 minutes.

10 μl of a test sample suspected to contain pyropheophytinase activity is added to the tube containing reaction buffer and substrate. The tube is incubated at 40° C. for 10 minutes with shaking at 900 rpm.

After 10 minutes incubation, 10 μl of the sample/reaction buffer/substrate mixture is transferred to another Eppendorf tube containing 1 ml of a detection solution comprising 0.015% Triton X-100, 15% isopropanol and 0.05M NaOH. Immediately the tube is closed and mixed on Whirley mixer for 5 seconds. 2 aliquots each of 200 μl of the diluted sample are transferred to a black microtiter plate. 10 minutes after the last sample is taken the samples are measured on a fluorescence plate reader at ex. 410 nm and em. 670 nm at room temperature (20° C. to 25° C.).

A standard curve may be constructed as described in Example 1 and used to determine pyropheophorbide concentrations in each sample after 10 minutes incubation at 40° C. For instance, the slope of the standard curve is relative fluorescence (RFU) as a function of pyropheophorbide concentration in μM. Therefore for a particular sample, [pyropheophorbide] (μM)=RFU/Slope of standard curve. Pyropheophytinase activity in the test sample may be calculated as the number of μmoles pyroheophorbide produced per minute of incubation at 40° C., taking into account the dilution of the sample in the assay.

### Example 5

### Pheophytinase Assay

Example 4 is repeated replacing pyropheophytin as substrate with pheophytin (at a concentration of 56 mM). A standard curve may be constructed as described in Example 2. Pheophytinase activity in the test sample may be calculated as the number of μmoles pheophorbide produced per minute of incubation at 40° C., taking into account the dilution of the sample in the assay.

### Example 6

### Chlorophyllase Assay

Example 4 is repeated replacing pyropheophytin as substrate with chlorophyll (at a concentration of 56 mM). A standard curve may be constructed using varying concentrations of chlorophyllide. Chlorophyllase activity in the test sample may be calculated as the number of μmoles chlorophyllide produced per minute of incubation at 40° C., taking into account the dilution of the sample in the assay.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry or related fields are intended to be within the scope of the following claims.

1. A method for detecting a phytol-free chlorophyll derivative in a sample, comprising a step of detecting a fluorescent signal associated with the phytol-free chlorophyll derivative, wherein a fluorescent signal associated with chlorophyll or a phytol-containing chlorophyll derivative in the sample is quenched, and wherein chlorophyll or the phytol-containing chlorophyll derivative is present in the form of an aqueous solution of dimers or other non-colloidal multimers.

2. The method of claim 1, wherein the fluorescent signal associated with chlorophyll or the phytol-containing chlorophyll derivative is quenched by dimerization of chlorophyll or the phytol-containing chlorophyll derivative.

3. The method of claim 1, wherein chlorophyll or the phytol-containing chlorophyll derivative dimerizes preferentially compared to the phytol-free chlorophyll derivative.

4. The method of claim 1, wherein the phytol-free chlorophyll derivative comprises pheophorbide or pyropheophorbide, or the phytol-containing chlorophyll derivative comprises pheophytin or pyropheophytin.

5. The method of claim 1, wherein the detecting step is performed in a detection solution comprising a surfactant, an alcohol or an alkali.

6. The method of claim 5, wherein the alcohol comprises isopropanol or ethanol.

7. The method of claim 6, wherein the detection solution comprises 12 to 20% by weight alcohol.

8. The method of claim 5, wherein the surfactant comprises 4-(1,1,3,3-tetramethylbutyl)phenyl-polyethylene glycol.

9. The method of claim 5, wherein the detection solution comprises 0.01 to 0.03% by weight surfactant.

10. The method of claim 1, wherein the detecting step is performed at 20 to 25° C.

11. The method of claim 1, wherein the detecting step is performed at a pH of greater than 10.0.

12. The method of claim 1, wherein a fluorescent signal of wavelength about 670 nm is detected.

13. An assay method for quantifying enzyme activity in a sample, wherein the enzyme is capable of hydrolyzing chlorophyll or a phytol-containing chlorophyll derivative, the assay method comprising:
a.) contacting the sample with chlorophyll or a phytol-containing chlorophyll derivative; and
b.) detecting production of a phytol-free chlorophyll derivative by detecting a fluorescent signal associated with the phytol-free chlorophyll derivative, wherein a fluorescent signal associated with chlorophyll or a phytol-containing chlorophyll derivative in the sample is quenched, and wherein chlorophyll or the phytol-containing chlorophyll derivative is present in the form of an aqueous solution of dimers or other non-colloidal multimers.

14. (canceled)

15. (canceled)

16. A kit for quantifying enzyme activity in a sample, wherein the enzyme is capable of hydrolyzing chlorophyll or a phytol-containing chlorophyll derivative, comprising:
a.) a reaction solution in which the enzyme is active;
b.) a substrate comprising chlorophyll or a phytol-containing chlorophyll derivative; and
c.) a detection solution in which the substrate dimerizes preferentially compared to a phytol-free chlorophyll derivative produced by the enzyme, and in which the substrate is present in the form of an aqueous solution of dimers or other non-colloidal multimers.

17. The kit of claim 16, wherein
(i) the reaction solution comprises a surfactant, acetone and a buffer, and
(ii) the substrate comprises pheophytin or pyropheophytin.

18. (canceled)

19. (canceled)

20. (canceled)

21. (canceled)

22. (canceled)

23. The method of claim 1, wherein the detection step is performed in the absence of liposomes.

24. The method of claim 5, wherein the surfactant comprises 4-(1,1,3,3-tetramethylbutyl)phenyl-polyethylene glycol, the alcohol comprises isopropanol and the alkali comprises sodium hydroxide.

25. The assay method of claim 13, wherein the enzyme is active during step (a) and the enzyme is inactive during step (b).

26. The method of claim 1, wherein the phytol-free chlorophyll derivative comprises pheophorbide or pyropheophorbide, and the phytol-containing chlorophyll derivative comprises pheophytin or pyropheophytin.

27. The method of claim 1, wherein the detecting step is performed in a detection solution comprising a surfactant, an alcohol and an alkali.
